(19) United States
(12) Patent Application Publication
(10) Pub. No.: US 2020/0138413 A1
(43) Pub. Date: May 7, 2020

(54) OBJECT INFORMATION ACQUIRING APPARATUS AND OBJECT INFORMATION ACQUIRING METHOD

(71) Applicant: CANON KABUSHIKI KAISHA, Tokyo (JP)

(72) Inventors: Mie Okano, Yokohama-shi (JP); Kenichi Nagae, Yokohama-shi (JP)

(21) Appl. No.: 16/668,221

(22) Filed: Oct. 30, 2019

(30) Foreign Application Priority Data
Nov. 2, 2018 (JP) 2018-207337

(51) Int. Cl. A61B 8/08; A61B 8/00; A61B 5/00; A61B 90/00

(52) U.S. Cl. CPC A61B 8/5238; A61B 8/5207; A61B 8/461; A61B 8/467; A61B 2090/3983; A61B 5/0073; A61B 5/0033; A61B 90/39; A61B 5/0095

(57) ABSTRACT

An object information acquiring apparatus includes: an optical image acquiring unit which acquires an optical image obtained by optically capturing an image of an object on which a marker is arranged; a photoacoustic image acquiring unit which acquires a photoacoustic image derived from a photoacoustic wave generated from the object irradiated with light; and a superimposed image generating unit which generates a graphic on the basis of positional information of the marker in the optical image and which generates a superimposed image by superimposing the graphic onto the photoacoustic image.

### BACKGROUND OF THE INVENTION

### Field of the Invention

The present invention relates to an object information acquiring apparatus and an object information acquiring method.

### Description of the Related Art

In recent years, research has been conducted on optical imaging in which an object is irradiated with light to acquire characteristic information of the object. In particular, attention has focused on a technique referred to as photoacoustic tomography (PAT) which utilizes a photoacoustic effect where irradiating an object with light causes a light absorber to generate an acoustic wave (a photoacoustic wave). A photoacoustic apparatus using photoacoustic tomography technology detects and analyzes a photoacoustic wave generated by an object due to the photoacoustic effect and acquires characteristic information indicating optical characteristic values inside the object. Examples of characteristic information include an initial sound pressure and an absorption coefficient. In addition, by focusing on hemoglobin contained in large amounts in blood as a light absorber, an absorption coefficient of oxyhemoglobin or deoxyhemoglobin or oxygen saturation in blood can be acquired. Furthermore, on the basis of such information, an image of a blood vessel distribution inside an object can be created.

Japanese Patent No. 5911196 describes a photoacoustic apparatus that acquires an optical image in addition to a photoacoustic image.

### SUMMARY OF THE INVENTION

However, in a photoacoustic apparatus that acquires a photoacoustic image and an optical image, it is sometimes difficult to assess a positional relationship between a photoacoustic image and an actual object. An example thereof is a case where a region of interest to be an observation object is not a location of which an outline is readily recognizable such as the palm of a hand but a location of which a body surface profile has only a small number of characteristic points such as a thigh, the trunk, or a breast. In such a case, it is difficult to access which position of the object a blood vessel image obtained in a photoacoustic image corresponds to.

The present invention has been made in consideration of such problems. An object of the present invention is to provide a technique that enables a positional relationship between a photoacoustic image and an object to be readily assessed in an object information acquiring apparatus using photoacoustic tomography.

The present invention adopts the following configuration. In other words,

an object information acquiring apparatus includes:

an optical image acquiring unit which acquires an optical image obtained by optically capturing an image of an object on which a marker is arranged;

a photoacoustic image acquiring unit which acquires a photoacoustic image derived from a photoacoustic wave generated from the object irradiated with light; and

a superimposed image generating unit which generates a graphic on the basis of positional information of the marker in the optical image and which generates a superimposed image by superimposing the graphic onto the photoacoustic image.

The present invention also adopts the following configuration. In other words,

an object information acquiring method includes:

acquiring an optical image obtained by optically capturing an image of an object on which a marker is arranged;

acquiring a photoacoustic image derived from a photoacoustic wave generated from the object irradiated with light; and

generating a graphic on the basis of positional information of the marker in the optical image and generating a superimposed image by superimposing the graphic onto the photoacoustic image.

According to the present invention, a technique that enables a positional relationship between a photoacoustic image and an object to be readily assessed in an object information acquiring apparatus using photoacoustic tomography can be provided.

Further features of the present invention will become apparent from the following description of exemplary embodiments with reference to the attached drawings.

### BRIEF DESCRIPTION OF THE DRAWINGS

FIG. 1 is a block diagram showing a configuration of an object information acquiring apparatus according to a first embodiment;

FIGS. 2A to 2C are diagrams showing a situation where a marker is arranged on an object;

FIG. 3 is a diagram showing a measurement location of an object and a marker according to the first embodiment;

FIG. 4 is a diagram showing an optical image according to the first embodiment;

FIG. 5 is a diagram showing a photoacoustic image according to the first embodiment;

FIG. 6 is a diagram showing a superimposed image according to the first embodiment;

FIG. 7 is a flow chart showing a flow of processes of the first embodiment;

FIG. 8 is a diagram showing a display screen according to a second embodiment;

FIG. 9 is a diagram showing a modification of a display screen; and

FIGS. 10A and 10B are diagrams showing a display screen according to a third embodiment.

### DESCRIPTION OF THE EMBODIMENTS

Hereinafter, preferred embodiments of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the description presented below.

The present invention relates to a technique for irradiating an object with an electromagnetic wave such as light, performing information processing on an acoustic wave generated from inside the object and acquiring characteristic information of the inside of the object (object information), and displaying the object information. Therefore, the present invention can be considered a photoacoustic apparatus or a control method thereof, an object information acquiring apparatus or a control method thereof, an object information acquiring method, an information processing apparatus or a control method thereof, a signal processing method, an information processing method, or a display method. The present invention can also be considered a program that causes an information processing apparatus including hardware resources such as a CPU and a memory to execute the respective methods described above or a storage medium storing the program. The storage medium may be a computer-readable non-transitory storage medium.

Characteristic information in a photoacoustic apparatus refers to a value reflecting an absorption amount or an absorption rate of optical energy corresponding to each of a plurality of positions inside the object which is generated using a received signal derived from a photoacoustic wave. For example, the characteristic information includes a generation source of acoustic waves generated by irradiation of light with a single wavelength, initial sound pressure inside an object, or a light energy absorption density or an absorption coefficient derived from the initial sound pressure. In addition, a concentration of a substance constituting tissue can be acquired from characteristic information derived from a plurality of mutually different wavelengths. By obtaining an oxyhemoglobin concentration and a deoxyhemoglobin concentration as concentrations of substances, a distribution of oxygen saturation can be acquired. Furthermore, as concentrations of substances, a glucose concentration, a collagen concentration, a melanin concentration, a volume fraction of fat or water, and the like can also be acquired.

A two-dimensional or three-dimensional characteristic information distribution is obtained based on characteristic information at each position in the object. Distribution data may be generated as image data. Characteristic information may be obtained as distribution information such as an initial sound pressure distribution, an energy absorption density distribution, an absorption coefficient distribution, or an oxygen saturation distribution. Data indicating distribution information is also referred to as photoacoustic image data, ultrasonic image data, or reconstructed image data.

An acoustic wave as referred to in the present invention is typically an ultrasonic wave and includes an elastic wave which is also called a sonic wave or an acoustic wave. An electrical signal transformed from an acoustic wave by a transducer or the like is also referred to as an acoustic signal. However, descriptions of an ultrasonic wave and an acoustic wave in the present specification are not intended to limit a wavelength of the elastic waves thereof. An acoustic wave generated by a photoacoustic effect is referred to as a photoacoustic wave or an optical ultrasonic wave. An electric signal derived from a photoacoustic wave is also referred to as a photoacoustic signal.

The object information acquiring apparatus according to the present invention is suitable for diagnosing a vascular disease, a malignant tumor, or the like and performing a follow-up of chemotherapy of a human or an animal. Examples of an object include a part of a living organism such as a breast or a hand of an examinee, an animal other than a human such as a mouse, a non-living object, and a phantom. As an object of imaging by a photoacoustic technique, in addition to a light absorber constituting an object interior or an object surface, an introduced contrast agent can also be used.

The object information acquiring apparatus according to the present invention can be preferably applied to a photoacoustic apparatus for acquiring a photoacoustic image and an optical image. The technique according to the present invention enables a positional relationship between a photoacoustic image and an optical image and a correspondence between a photoacoustic image and a position of an object to be assessed in a favorable manner. As a result, a physician, a technician, or the like can refer to a photoacoustic image to identify a position of a blood vessel in a region of interest of an object and perform a procedure for collecting a skin flap or the like in a favorable manner.

In order to access a positional relationship between a photoacoustic image and an optical image, a method is conceivable in which a photoacoustic image and an optical image are acquired after arranging a marker constituted by a light absorber in a region of interest of an object in advance. Accordingly, after acquiring the photoacoustic image and the optical image, both images can be displayed in superposition by using the marker as a guide.

However, with this method, a skin color or a characteristic point on an object surface ends up being reflected in the superimposed image and may interfere with a user's reading of the image. In addition, with this method, a light absorber having absorption characteristics in a wavelength of light used in photoacoustic measurement is used as the marker. Accordingly, while there is an advantage that a position of the marker can be more readily assessed, there is a risk that noise derived from the marker may appear in the photoacoustic image.

The object information acquiring apparatuses according to the respective embodiments described below were conceived as a result of studies carried out by the present inventors in consideration of the circumstances described above.

### First Embodiment

It should be noted that, in the respective embodiments described below, same components are generally assigned same reference characters and a description thereof will be simplified. It is assumed that the object information acquiring apparatus according to the present embodiment is to be used during a procedure of collecting a perforator flap. Generally, a Doppler blood flow meter is used to check a position of a penetrating branch. However, since photoacoustic measurement is effective in assessing a blood vessel position as described above, performing a photoacoustic measurement and acquiring a photoacoustic image prior to a procedure of collecting a perforator flap is being considered. Accordingly, it is expected that a user such as a physician, a technician, or an image reader will be able to assess a position or a running condition of a blood vessel (in particular, a penetrating branch) and obtain information helpful in determining a position and a range of a skin flap to be collected.

While viewing a photoacoustic image, the user assesses which portion of the object a blood vessel image in the photoacoustic image corresponds to and which location of the object constitutes the region of interest. In addition, the user performs a procedure while referring to the position of the penetrating branch in the photoacoustic image. However, a location which is relatively wide, and which does not have characteristics on a body surface such as a thigh or the trunk is used for collecting a skin flap. Therefore, it is difficult to assess a positional relationship between the photoacoustic image and the actual object. In consideration thereof, in the present embodiment, in order to facilitate assessment of the positional relationship between a photoacoustic image and an object, a position of the region of interest in the photoacoustic image is displayed to the user in an easily understood manner. Accordingly, the user can readily find a penetrating branch. As a result, the time required to search for a penetrating branch and determine a position and a range of a skin flap to be collected can be reduced.

Configuration of Apparatus

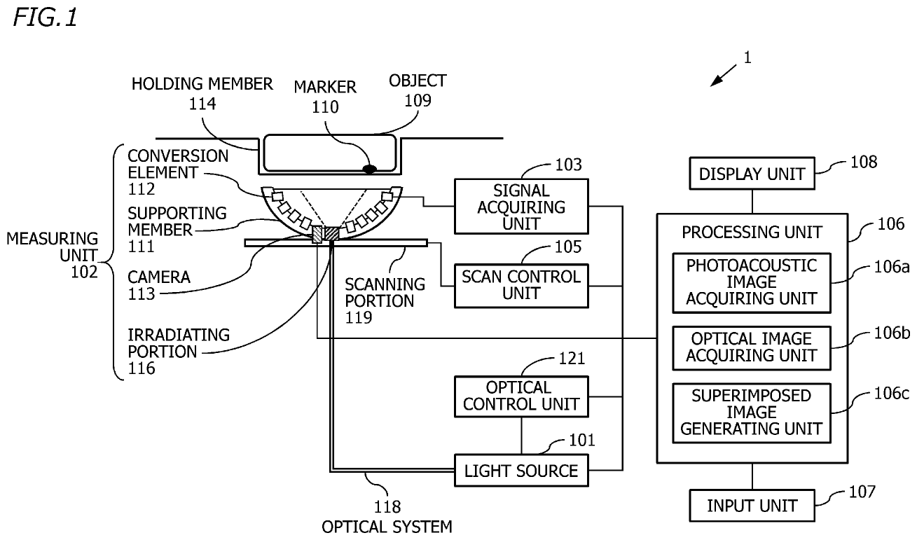

FIG. 1 is a block diagram illustrating a configuration of an object information acquiring apparatus 1 according to the present embodiment. The object information acquiring apparatus has a light source 101, a measuring unit 102, a signal acquiring unit 103, a scan control unit 105, a processing unit 106, an input unit 107, a display unit 108, a scanning unit 119, an optical control unit 121, and the like. Hereinafter, each component will be described.

Light Source

The light source 101 is an apparatus that generates pulse light for irradiating an object 109. While the light source 101 is desirably a laser light source in order to obtain a large output, a light-emitting diode, a flash lamp, or the like may also be used in place of a laser. When using a laser as the light source, various lasers such as a solid-state laser, a gas laser, a dye laser, and a semiconductor laser can be used. Timings, waveforms, intensity, and the like of irradiation are controlled by the optical control unit 121. The optical control unit may be integrated with the light source 101. While an alexandrite laser is used as the light source 101 in the present embodiment, a YAG laser or a titanium sapphire laser may be used instead. In addition, the light source 101 may constitute a part of the object information acquiring apparatus or the object information acquiring apparatus may cooperate with an external light source to irradiate light.

Desirably, a wavelength of the pulse light is a specific wavelength which is absorbed by a specific component among components constituting the object 109 and which enables light to propagate to the inside of the object. Specifically, in a case where the object 109 is a living organism, light with a wavelength of at least 650 nm and not more than 1100 nm is desirably used. Moreover, in a case where oxygen saturation is acquired as object information, two or more wavelengths may be used.

In addition, in order to change an irradiation wavelength in accordance with an absorption coefficient of a light absorber to be an observation object, a wavelength-variable light source may be used. When using a wavelength-variable light source, after determining a material of a marker 110, a wavelength at which the absorption coefficient of the marker 110 is lower than the absorption coefficient of the object 109 and the marker 110 is less likely to be reflected in a photoacoustic image may be selected.

Furthermore, in order to effectively generate a photoacoustic wave, light must be irradiated within a sufficiently short period of time in accordance with thermal characteristics of the object 109. In a case where the object 109 is a living organism, a pulse width of the generated pulse light is preferably around 10 to 50 nanoseconds.

Light emitted from the light source 101 is transmitted via an optical system 118 to an irradiating unit 116 and subsequently irradiates the object 109. The optical system 118 and the irradiating unit 116 are constituted by an optical component such as a lens or a mirror or an optical waveguide such as an optical fiber. Due to the optical system 118 and the irradiating unit 116, pulsed light is processed into a prescribed light distribution profile and guided to the object 109.

Optical Control Unit

The optical control unit 121 controls the light source 101 and the irradiating unit 116 to control an irradiation timing of light, a waveform of light, an intensity of light, a pulse length or a repetition frequency of pulsed light, and the like. The optical control unit 121 performs optical control by transmitting control signals to the light source 101 and the irradiating unit 116 on the basis of control information input by the user, a program and control information stored in a memory in advance, and the like. As the optical control unit 121, an information processing apparatus which includes computing resources such as a processor, a memory, and communicating means and which operates in accordance with a program or the like can be used. In addition, the processing unit 106 may double as an optical control unit. The optical control unit 121 stores information regarding an irradiation timing of pulsed light in a memory. In a case where a light irradiation position moves during measurement, the optical control unit 121 stores information regarding an irradiation position at each irradiation timing of the pulsed light in the memory.

Measuring Unit

The measuring unit 102 is a unit which irradiates the object 109 with light and which receives a photoacoustic wave generated from the object 109. The measuring unit 102 is structured such that a plurality of conversion elements 112 are arranged on an inner surface of a supporting member 111 which is a hemispherical member. In addition, the irradiating unit 116 and a camera 113 are arranged in a bottom unit of the supporting member 111.

The measuring unit 102 causes a relative positional relationship between the measuring unit 102 and an object to change by being driven by the scan control unit 105 and the scanning unit 119. Performing acoustic wave measurement while performing scanning in this manner enables photoacoustic waves generated from various positions of the object to be received. As a result, the object can be measured in a short amount of time. As the supporting member 111, a material having a certain degree of strength such as a metal or a resin is favorably used. An inside of the supporting member 111 may be filled with an acoustic matching medium in order to facilitate transmission of acoustic waves. Examples of the acoustic matching medium include water and gels. In addition, the entire measuring unit 102 may be positioned inside a liquid vessel capable of holding an acoustic matching medium.

The conversion element 112 is a unit which receives an acoustic wave propagating from inside of an object or an object surface and converts the acoustic wave into an electrical signal. Desirably, the conversion element 112 has high sensitivity and a wide frequency band. Favorable examples of the conversion element 112 include a piezoelectric element using lead zirconate titanate (PZT) or the like, a capacitive micromachined ultrasonic transducer (CMUT), and an element using a Fabry-Perot interferometer. Since a frequency band of acoustic waves generated by a living organism mainly range from 100 kHz to 100 MHz, an element capable of receiving this frequency band is favorably used as the conversion element 112.

Using a plurality of conversion elements 112 as described above enables acoustic waves to be simultaneously received at a plurality of positions. As a result, since measurement time can be reduced, an effect of vibration of an object or the like is reduced. In addition, an SN ratio can be improved. When using the illustrated hemispherical supporting member 111, a high sensitivity region where directional axes of the respective conversion elements concentrate can be provided near a center of the hemisphere. Alternatively, the plurality of conversion elements 112 may be arranged in a linear or two-dimensional array. In addition, a single element measuring unit may be used.

A region that can be imaged by the measuring unit 102 by one light irradiation is determined in accordance with an apparatus configuration and settings. In a case where the region of interest desired by the user is wider than an imageable region, a photoacoustic image of the entire region of interest can be acquired by repetitively performing photoacoustic measurement while having the scanning unit 119 scan the measuring unit 102.

Signal Acquiring Unit

The signal acquiring unit 103 subjects an electric signal acquired by a conversion element to an amplification process or a digital conversion process and outputs a photoacoustic signal. The signal acquiring unit 103 is typically constituted by an amplifier, an A/D converter, a field programmable gate array (FPGA) chip, or the like. In a case where a plurality of signals are obtained from the conversion elements 112, desirably, a plurality of signals can be processed simultaneously. In addition, the signal acquiring unit 103 may perform a correction process on signals.

Alternatively, the signal acquiring unit 103 may double as a reception control unit that performs reception control of an acoustic wave and a photoacoustic signal. However, the processing unit 106 may doubly provide a function of a reception control unit.

In the description given above, only the object 109 has been described as an object which the object information acquiring apparatus irradiates with light. However, in reality, light emitted from the light source 101 irradiates not only the object 109 but also the marker 110. In addition, the conversion element 112 receives not only a photoacoustic wave derived from the object but also a photoacoustic wave derived from the marker. Nevertheless, as long as an intensity of a photoacoustic wave derived from the marker is sufficiently lower than an intensity of a photoacoustic wave derived from the object with respect to a wavelength of the irradiation light, processes of the present embodiment can be executed. Typically, the intensity of a photoacoustic wave derived from the marker is favorably equal to or lower than one tenth of the intensity of a photoacoustic wave derived from the object. In addition, when determining a wavelength of the irradiation light or a material of the marker, the wavelength or the material is favorably selected so as to satisfy such conditions.

Scan Control Unit and Scanning Unit

The scan control unit 105 controls the scanning unit 119 to change a relative positional relationship between the measuring unit 102 and an object. The scanning unit 119 is constituted by a driving mechanism such as a drive stage or a ball screw and a power source such as a motor. In the case of two-dimensional scanning, the scan control unit 105 moves the supporting member 111 within a movement plane below the object. Any movement path such as a spiral scan or a raster span may be adopted. In addition, photoacoustic measurement (light irradiation and reception of a photoacoustic signal) is repeated at constant periods during the scan.

The scan control unit 105 controls a scan start time, a scan end time, a scan path, a movement speed, and the like when controlling the scanning unit 119. The scan control unit 105 performs scan control by transmitting control signals to the scanning unit 119 on the basis of control information input by the user, a program and control information stored in a memory in advance, and the like. As the scan control unit 105, an information processing apparatus which includes computing resources such as a processor, a memory, and communicating means and which operates in accordance with a program or the like can be used. In addition, the processing unit 106 may double as a scan control unit.

The scan control unit 105 transmits positional information of the measuring unit 102 during an event related to photoacoustic measurement such as an irradiation position during pulsed light irradiation and an image capturing position during acquisition of an optical image to the processing unit 106 in association with time information that enables a timing to be identified.

As a result of an optical image acquiring unit 106b, the scan control unit 105, the optical control unit 121, and a signal acquiring unit (a reception control unit) working in conjunction with one another, an irradiation position and an irradiation timing of light, a reception position and a reception timing of a photoacoustic wave, and an image capturing position and an image capturing timing of an optical image are stored. Using these pieces of information, the processing unit 106 can perform a joining process of optical images, a joining process of photoacoustic images, and an assessment of a positional relationship between an optical image and a photoacoustic image.

Processing Unit

The processing unit 106 includes a photoacoustic image acquiring unit 106a, the optical image acquiring unit 106b, and a superimposed image generating unit 106c.

Photoacoustic Image Acquiring Unit

The photoacoustic image acquiring unit 106a processes a photoacoustic signal generated by the signal acquiring unit 103 and generates an image representing characteristic information inside an object. While methods of reconstruction include a Fourier transform method, a universal back-projection method (UBP method), and a filtered back-projection method, any method may be used. The photoacoustic image acquiring unit may acquire an absorption coefficient distribution by obtaining a light amount distribution inside the object from a light amount of irradiation light. The photoacoustic image acquiring unit may calculate functional information such as oxygen saturation inside the object by processing a photoacoustic signal obtained through irradiation of irradiation light with a plurality of wavelengths. The photoacoustic image acquiring unit may perform image processing for converting a reconstructed photoacoustic image into a format suitable for a comparison process with an optical image, a superposition process with an optical image, a display process to the user, and the like.

Optical Image Acquiring Unit

The optical image acquiring unit acquires an optical image of the object and the marker 110 by working in conjunction with the camera 113. The camera 113 is a visible light camera that optically captures an image of a surface of the object 109. However, the camera 113 may be any type of camera as long as information on the surface of the object 109 can be acquired. For example, the camera 113 may be an image capturing apparatus that uses light in the infrared range instead of the visible light range. In the present embodiment, the camera 113 capable of capturing an entire region of interest is arranged at a position opposing the object 109 on the supporting member 111. The optical image acquiring unit receives an optical image signal from the camera 113 and stores the optical image signal as image data. The image data may be moving image data. Hereinafter, an image captured by the optical image acquiring unit will be referred to as an optical image.

It should be noted that the camera 113 need not be arranged on the supporting member 111. In this case, a configuration may be adopted which causes the measuring unit 102 to retreat from an optical image capturing range of the camera 113 when acquiring an optical image.

In a case where a region of which an optical image can be captured by one optical image capturing operation is narrower than the region of interest, the optical image acquiring unit generates an optical image of the entire region of interest by joining together a plurality of optical images. When joining optical images in a configuration in which the camera 113 is mounted to the measuring unit 102 as shown in FIG. 1, position control information of the measuring unit 102 held by the scan control unit 105 and image capturing timing information of an optical image held by the optical control unit 121 are used.

The optical image acquiring unit further performs image processing on an optical image obtained from a signal from the camera 113. Specifically, positional information of the marker 110 is detected from the optical image. For the detection of positional information of a marker, a method of detecting a characteristic point image of a color or a shape of the marker 110 or a method of using a marker position designated by the user while looking at an image displayed on the display unit can be used. Known tracking methods may also be used. In addition, in a similar manner to the photoacoustic image acquiring unit, the optical image acquiring unit may also perform image processing for converting an optical image generated by the optical image acquiring unit into a format suitable for a comparison process with a photoacoustic image, a superposition process with a photoacoustic image, a display process to the user, and the like.

The superimposed image generating unit performs image processing such as a compositing process or a superposition process which is necessary for display on the display unit 108 to be described later. For example, on the basis of the positional information of the marker 110, the superimposed image generating unit generates a superimposed image which displays a graphic (item) indicating the position of the marker on a photoacoustic image generated by the photoacoustic image acquiring unit and displays the superimposed image on the display unit 108.

In addition, the processing unit 106 may have a functional block for receiving various instructions input via the display unit 108 and the input unit 107. Examples of instructions include those related to setting or changing a measurement parameter, starting or ending a measurement, acquiring an optical image, selecting a processing method of an image, selecting a display method of a marker, storing patient information and an image, and analyzing data.

The processing unit 106 may be constituted by a computer having a CPU, a RAM, a nonvolatile memory, and a control port. Control is performed as a program stored in the nonvolatile memory (a storage unit) is executed by the CPU. The processing unit 106 may be realized by a general-purpose computer or an exclusively designed workstation. In addition, units that serve a calculation function may be constituted by a processor such as a CPU or a GPU or an arithmetic circuit such as an FPGA chip. Such units may not only be constituted by a single processor or a single arithmetic circuit but may also be constituted by a plurality of processors or a plurality of arithmetic circuits. Furthermore, the processing unit 106 may be provided with a non-transitory storage medium such as a ROM, a magnetic disk, or a flash memory or a volatile medium such as a RAM.

It should be noted that components such as the processing unit 106, the scan control unit 105, the optical control unit 121, and the signal acquiring unit 103 may be each configured as a separate body as illustrated or may be mounted as functional blocks of a same information processing apparatus. In addition, each functional block included in the processing unit 106 may be mounted to a same information processing apparatus as illustrated or may be mounted to different information processing apparatuses and operate in conjunction with each other. The functional blocks can be configured in any way as long as the object information acquiring apparatus as a whole is capable of executing processes according to the present embodiment.

Input Unit

The input unit 107 is an apparatus used by a user for inputting instruction information. For example, while a pointing device such as a mouse, a trackball, or a touch panel, a keyboard, or the like can be used as the input unit 107, but the input unit 107 is not limited thereto.

Display Unit

The display unit 108 is an apparatus which displays information acquired by the processing unit 106 and processed information based on the acquired information. For example, a liquid crystal display, a plasma display, an organic EL display, an FED, or the like can be used as the display unit 108. A projector can also be used as the display unit 108. The projector may project an image onto a screen. Alternatively, a positioned image may be directly projected onto an object from the projector. The display unit 108 may be provided separate from the photoacoustic apparatus. For example, an acquired photoacoustic image, optical image, or superimposed image may be transmitted to an external display unit 108 in a wired or wireless manner.

Holding Member

The holding member 114 is a member that holds an object that is a measurement object. A material having characteristics for transmitting light and acoustic waves such as a polyethylene terephthalate or acrylic is preferably used as the holding member 114. In addition, the holding member 114 is preferably strong enough to support the object 109. In order to secure strength, a holding member with a prescribed thickness or more or a rib-like or mesh-like supporting member may be used. Furthermore, a space between the object 109 and the holding member 114 may be filled with an acoustic matching medium in order to facilitate transmission of acoustic waves. Examples of the acoustic matching medium include water and gels.

The object information acquiring apparatus is preferably equipped with a housing for storing at least the measuring unit 102 among the respective components described above. The housing is preferably strong enough to support the body of an examinee. For example, a bed-like examinee supporting surface may be provided on an upper surface of the housing. In this case, an opening into which the object that is a part of the examinee is to be inserted is preferably provided on the examinee supporting surface. For example, in a case where the object is a thigh or a breast, the examinee lies down on the examinee supporting surface in a prone position and inserts the object into the opening. In addition, when using a holding member for holding the object, it is also preferable to provide a fixing member for fixing the holding member to an edge of the opening.

Process Flow

A flow of processes according to the present embodiment will be described with reference to the flow chart shown in FIG. 7.

In step S101, the user sets a measurement parameter and a region of interest via the input unit. Measurement parameters may include any parameter such as a setting value related to irradiation light, a setting value related to scanning, a setting value related to a photoacoustic image, or a setting value related to display. For example, parameters related to operations of the apparatus include various pieces of information related to the creation of a superimposed image such as a path or a speed of scanning by the scan control unit 105, an intensity or an interval of light irradiation during photoacoustic measurement, an image quality of a photoacoustic image or an optical image, and information on the marker 110. A region of interest may be designated by numerical values, selected from regions set by default, or designated by a stylus or the like while looking at a screen of the display unit.

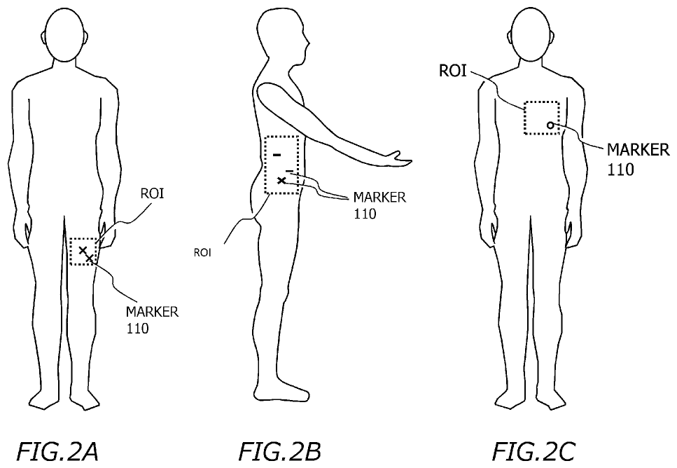

In step S102, the user installs the marker 110 in the region of interest. FIGS. 2A to 2C show situations where a marker has been installed in the region of interest (ROI). A region of interest is, respectively, set on a thigh in FIG. 2A, on the trunk in FIG. 2B, and on a breast in FIG. 2C.

FIG. 2A shows a situation where a marker that is an X mark has been arranged at a position of a penetrating branch inside the region of interest. In the present embodiment, the user performs a preparatory measurement using a Doppler blood flow meter to confirm the position of the penetrating branch and arranges a marker at the position. Alternatively, when determining the position of a marker, the user may determine a position of interest of a blood vessel or the like based on an anatomical guide on the surface of the object. For example, since the position of a penetrating branch is more or less determined for each skin flap, the position can be estimated based on an anatomical guide on the body surface.

When arranging a plurality of markers 110, markers 110 of a same type may be used or a combination of markers 110 of different types may be used. For example, markers 110 of different types may be respectively used for a penetrating branch and a normal blood vessel. For example, in FIG. 2B to be described later, a marker that is an X mark similar to that in FIG. 2A is arranged at the position of a penetrating branch. On the other hand, a linear marker is arranged at a position of interest having been estimated based on an anatomical guide on the body surface.

In addition, in FIG. 2C, a circular marker 110 corresponding to a position of interest inside the region of interest is arranged for purposes of diagnosis and follow-up. The shape of the marker 110 can be appropriately selected in accordance with an application or a size of an object region. For example, in addition to the shapes shown in FIGS. 2A to 2C, a marker may be a matrix, a radial shape, a triangle, or a rectangle.

The marker 110 is preferably confirmable in an optical image but substantially not drawn into a photoacoustic image. In other words, as the marker 110, a material that substantially does not have absorption characteristics in the wavelength used in photoacoustic measurement is used. Accordingly, noise in a photoacoustic image can be reduced. At least an intensity of an acoustic wave generated from the marker is preferably set to a level that is substantially negligible with respect to an intensity of an acoustic wave generated from a light absorber inside the object.

For example, in a case where irradiation light with a wavelength of 797 nm is used in photoacoustic measurement, red ink with a low absorption rate in the wavelength may be used as the marker 110. In addition, in a case where irradiation light with a wavelength of 850 nm is used in photoacoustic measurement, violet ink with a low absorption rate in the wavelength may be used as the marker 110. In this manner, a color or a type of the used marker 110 is preferably selected in accordance with a wavelength that is photographed in photoacoustic measurement. For example, violet ink does not appear in a photoacoustic image derived from the 850-nm wavelength but appears in a photoacoustic image derived from the 797-nm wavelength.

It should be noted that reference herein to the wavelength of 797 nm is not intended to strictly limit a wavelength to the numerical value but is described as a representative of wavelengths centered on 797 nm at which red ink exhibits similar absorption characteristics. A similar logic applies to the relationship between the wavelength of 850 nm and violet ink.

In the present embodiment, the user directly writes the marker on the object with a pen by using a selected ink.

It should be noted that the marker 110 is not limited to ink. Any marker that is drawn in an optical image but not drawn in a photoacoustic image can be used as the marker 110. For example, a sticker may be used as the marker 110 or a marker can be pasted to the object using an adhesive.

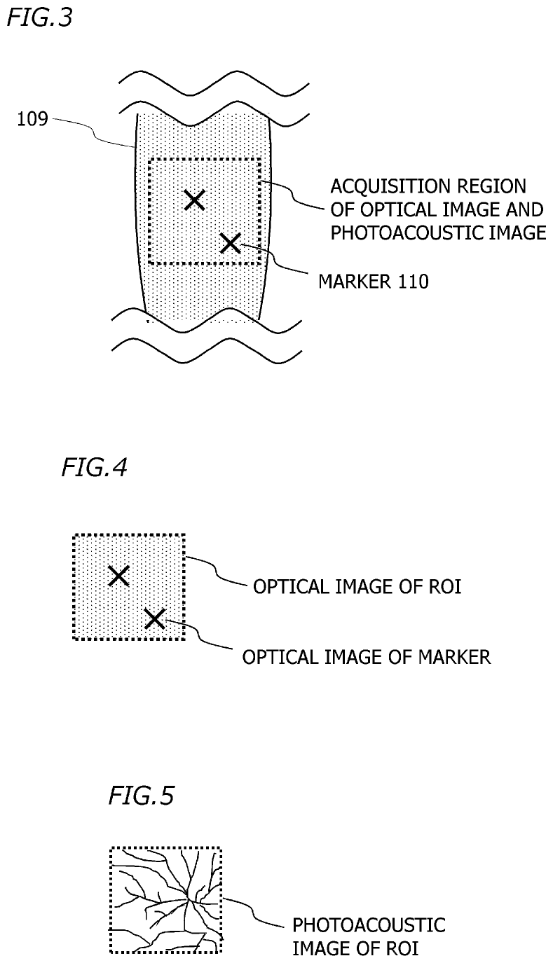

FIG. 3 is an enlarged view of the thigh shown in FIG. 2A. The diagram shows a region of interest to be an acquisition object of an optical image and a photoacoustic image and a marker arranged in the region of interest. In FIG. 3, a color of a skin surface of the object is depicted by hatchings. In addition, in the present embodiment, a photoacoustic image and an optical image are photographed in a same field of view and an optical image acquisition region and a photoacoustic image acquisition region coincide with each other. Furthermore, both the optical image acquisition region and the photoacoustic image acquisition region coincide with the region of interest.

In step S103, the optical image acquiring unit performs optical image capturing by controlling the camera 113 and acquires an optical image in which the surface of the object 109 and an image of the marker 110 is reflected. In a case where the region of interest is wide, the scan control unit 105 controls the scanning unit 119 and acquires a wide-range optical image.

FIG. 4 shows the optical image of the region of interest acquired in S103. In FIG. 4, the marker is drawn as an optical image. In addition, the color of the skin of the object is also drawn.

In step S104, photoacoustic measurement is performed. The optical control unit 121 controls the light source 101 and the irradiating unit 116 to irradiate the object with light. Accordingly, photoacoustic waves are generated from inside the object and from the object surface. The signal acquiring unit 103 receives a photoacoustic signal output from the conversion element 112 having detected a photoacoustic wave propagating from the object. The photoacoustic image acquiring unit 106a performs an image reconstruction process on the basis of the photoacoustic signal output from the signal acquiring unit 103 and positional information output from the scan control unit 105. Accordingly, a photoacoustic image is acquired as image data indicating a characteristic information distribution. In a case where the region of interest is wide, the scan control unit 105 controls the scanning unit 119 and acquires a wide-range photoacoustic image.

FIG. 5 shows the photoacoustic image of the region of interest acquired in S104 which depicts how blood vessels run.

It should be noted that either of the optical image capturing in S103 and the photoacoustic measurement in S104 may be performed first or the optical image capturing in S103 and the photoacoustic measurement in S104 may be performed in parallel to each other.

In step S105, the optical image acquiring unit 106b extracts positional information of the marker 110 from the optical image. In the present embodiment, the user designates a position of the marker 110 of the optical image using the input unit 107 while looking at the optical image displayed on the display unit 108. The optical image acquiring unit 106b acquires two-dimensional coordinates of the designated marker position and adopts the coordinates as marker position information.

A method of acquiring positional information of a marker is not limited to having the user designate a position of the marker. For example, the user may designate only a position of one marker, and the optical image acquiring unit 106b may read RGB values of the designated marker and extract other markers using the RGB values.

In addition, the optical image acquiring unit may perform a color extraction process using multi-level thresholding. In this case, the optical image acquiring unit performs color extraction from three attributes (HSV) of color, sets upper and lower thresholds with respect to respective primary color components (RGB) of color or the three attributes (HSV) of color of the marker 110, and extracts an image satisfying conditions of all thresholds.

Furthermore, a method of cutting an unnecessary background image and detecting only the marker 110 may also be used. In this case, the user sets an upper limit value and a lower limit value of concentrations to be cut as a background while viewing an optical image. By erasing an image equal to or lower than the lower limit value and displaying an image equal to or higher than the upper limit value by converting into brightness of the upper limit value, an optical image only showing the marker 110 can be acquired.

In step S106, the superimposed image generating unit 106c generates a superimposed image which displays a graphic based on positional information of the marker in superposition on a photoacoustic image. In addition, the superimposed image generating unit 106c stores the superimposed image in a memory and, at the same time, displays the superimposed image on the display unit 108. In the present embodiment, an extracted optical image of the marker that is an X marks is used without modification as the graphic. When creating the superimposed image, the superimposed image generating unit performs positioning of the photoacoustic image and the image in the photoacoustic image. Performing the positioning requires that a positional relationship between the photoacoustic image and the optical image be assessed and, to this end, information related to a field of view of each image, coordinate information related to control by the scan control unit, information related to timings of light irradiation and photoacoustic wave reception, and information related to timings of optical image capturing can be used.

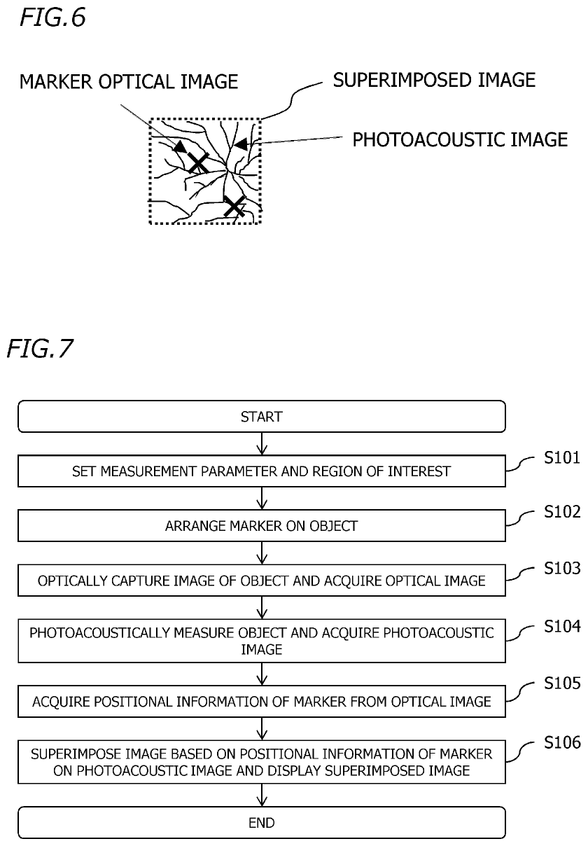

FIG. 6 shows the superimposed image of the region of interest acquired in S105. By superimposing only an extracted marker optical image as in the present embodiment, since the color of the skin of the object is prevented from hiding a photoacoustic image, the user can read the photoacoustic image with greater ease. Accordingly, the user can readily assess which portion of the photoacoustic image the region of interest corresponds to and analyze the portion in depth.

### Second Embodiment

An object information acquiring apparatus according to the present embodiment is similar to that according to the first embodiment shown in FIG. 1. In addition, a process flow according to the present embodiment is basically similar to that according to the first embodiment shown in FIG. 7. Hereinafter, portions which differ from the first embodiment will be mainly described.

When setting the region of interest in step S101, in the present embodiment, it is assumed that fields of view differ between a photoacoustic image and an optical image.

The object 109 according to the present embodiment is a trunk such as that shown in FIG. 2B. When installing a marker in step S102, the user arranges a different marker 110 in accordance with a difference in positions of interest inside the region of interest. In other words, the user arranges a marker that is an X mark at a position of a penetrating branch and arranges a linear marker at a position of interest having been estimated based on an anatomical guide on the body surface.

The subsequent steps S103 to S105 are performed in a similar manner to the first embodiment. In the present embodiment, since shapes of the region of interest differ between photoacoustic measurement and optical image capturing, shapes of a photoacoustic image and an optical image also differ.

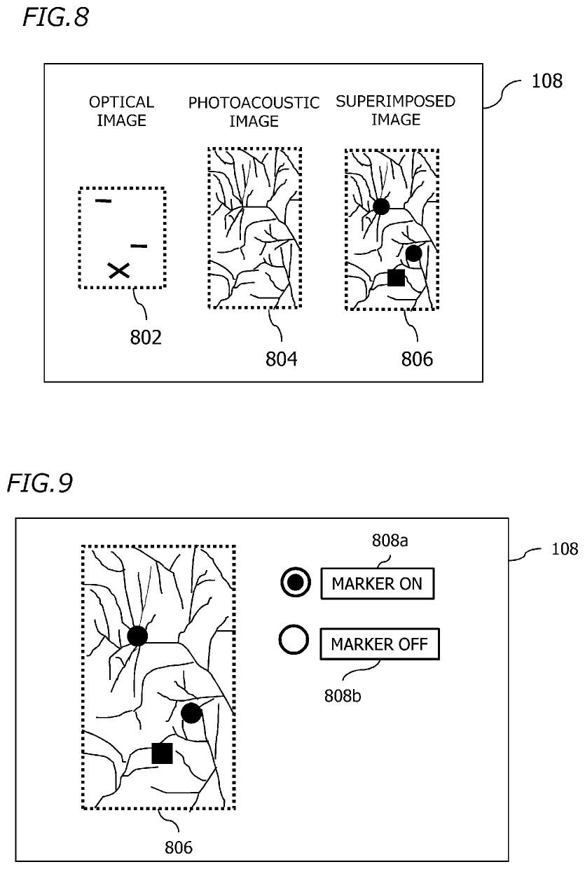

An image is displayed in S106. FIG. 8 shows an example of an image displayed on the display unit 108. In this example, an optical image 802, a photoacoustic image 804, and a superimposed image 806 are arranged side by side. In addition, the optical image and the photoacoustic image have mutually different fields of view, and the field of view of the optical image is narrower.

As is apparent from looking at the superimposed image 806 in FIG. 8, the graphic superimposed on the photoacoustic image in the present embodiment differs in shape from the marker arranged on the object. Specifically, a square graphic is displayed at a position of the marker that is an X mark, and a circular graphic is displayed at a position of the linear marker. In this manner, when displaying a graphic on the superimposed image, a color or a shape of the marker 110 may be reproduced or a graphic that differs from the marker 110 may be used. A graphic may be of any format as long as the user can obtain information necessary for a skin flap operation. As a graphic, a guide designated by the user using the input unit 107 may be used. In addition, on the basis of a marker written by the user, the optical image acquiring unit may add supplementary information to a graphic and display the supplementary information together with the graphic. For example, the user may only enter end points of a blood vessel with a marker but a display image may also display a line connecting the end points.

As the optical image, an optical image acquired by the optical image acquiring unit may be displayed without modification or only a marker image extracted by the optical image acquiring unit 106b may be displayed. By displaying only a marker image as in FIG. 8, the user can read an image without being obstructed by the color of the object surface or by structures.

In addition, the respective images may be displayed side by side in one window or each image may be displayed in a different window.

Furthermore, instead of arranging the respective images side by side, it is also preferable to sequentially display the images at prescribed time intervals or to configure the images to be switchable by a physical button or an icon on a GUI.

In addition, a button or an icon for hiding a graphic when the graphic obstructs the reading of the superimposed image may be provided. FIG. 9 shows, as a GUI for display switching, an icon 808a for switching ON a marker display and an icon 808b for switching OFF the marker display. By selecting any of the icons using a radio button, the user can switch between displaying and hiding a graphic.

According to the present embodiment, the user can readily assess which portion of the photoacoustic image the region of interest corresponds to and the region of interest can be readily analyzed in accordance with the type of the region of interest. In particular, characteristics of a position of interest assessed by the user when arranging a marker can be expressed on a superimposed image.

### Third Embodiment

An object information acquiring apparatus according to the present embodiment is similar to that according to the first embodiment shown in FIG. 1. In addition, a process flow according to the present embodiment is basically similar to that according to the first embodiment shown in FIG. 7. Hereinafter, portions which differ from the first embodiment will be mainly described.

The object information acquiring apparatus described in the present embodiment is used for the purposes of performing photoacoustic measurement, diagnosing a malignant tumor, a vascular disease, or the like, performing a follow-up of chemotherapy, and the like. As shown in FIG. 2C, the object 109 is a breast.

Therefore, in step S102 according to the present embodiment, the user estimates a tumor position using a previously captured photoacoustic image and a previously captured optical image as references, and pastes a red sticker that is a marker on the surface of the breast of the examinee. A method by which the user determines an installation location of a marker is not limited thereto. For example, a tumor position may be determined using devices of other modality such as an ultrasonic apparatus, a CT, or an MRI. In addition, a position of a tumor may be predicted from a shape or a characteristic point of a breast. Furthermore, in a case where the purpose is a follow-up of chemotherapy or the like, a position of a marker during previous photography may be used as a reference.

It is difficult to eliminate completely an occurrence of a photoacoustic wave from a marker during photoacoustic measurement. However, in order to minimize noise appearing in a photoacoustic image, a marker with minimal absorption of light with a wavelength used in the photoacoustic measurement is preferably used. For example, an intensity of a photoacoustic wave generated from a marker is lowered by one digit or more than an intensity of an acoustic wave generated from the object 109.

To take hemoglobin as an example, an absorption coefficient of hemoglobin is approximately 0.3/mm to 0.9/mm. Therefore, an absorption coefficient of the marker 110 is set to, for example, at least 0.05/mm and not more than 0.1/mm. Accordingly, noise due to a photoacoustic wave generated from a marker can be reduced to a level where the user's reading of an image is substantially unaffected.

In S103 to S105, optical image capturing, photoacoustic measurement, and extraction of a marker from an optical image are performed. In the present embodiment, characteristics of a color of a marker that is a sticker to be drawn in an optical image is stored in advance in a memory on the basis of optical characteristics of the sticker and apparatus characteristics of a camera with which optical image capturing is to be performed. The optical image acquiring unit extracts only the marker 110 from the optical image using the saved information.

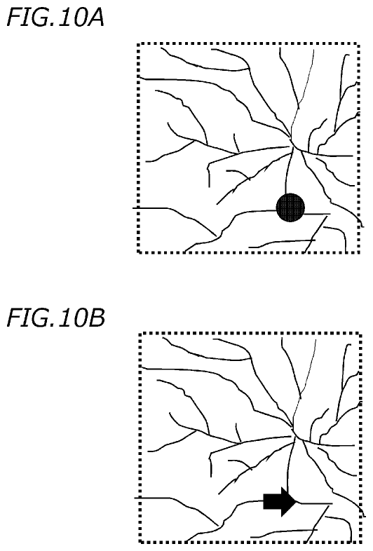

FIGS. 10A and 10B show examples of the superimposed image displayed in the present embodiment. In FIG. 10A, an optical image of an extracted marker (in this case, a sticker) is used as a graphic to be displayed in the superimposed image. In addition, in FIG. 10B, an arrow is displayed as a graphic at a position where the sticker is applied. A configuration may be adopted in which types of the graphic are switchable between FIGS. 10A and 10B in accordance with an instruction from the user via the input unit. Even according to the present embodiment, an image reader can readily assess which portion of the photoacoustic image the region of interest corresponds to and the region of interest can be readily analyzed in accordance with the type of the region of interest.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-207337, filed on Nov. 2, 2018, which is hereby incorporated by reference herein in its entirety.

What is claimed is:

1. An object information acquiring apparatus, comprising:
an optical image acquiring unit which acquires an optical image obtained by optically capturing an image of an object on which a marker is arranged;
a photoacoustic image acquiring unit which acquires a photoacoustic image derived from a photoacoustic wave generated from the object irradiated with light; and
a superimposed image generating unit which generates a graphic on the basis of positional information of the marker in the optical image and which generates a superimposed image by superimposing the graphic onto the photoacoustic image.

2. The object information acquiring apparatus according to claim 1, wherein
the optical image acquiring unit extracts an image of the marker from the optical image and extracts positional information, and
the superimposed image generating unit performs positioning of the photoacoustic image and the optical image and superimposes the graphic at a position corresponding to a position of the marker in the photoacoustic image.

3. The object information acquiring apparatus according to claim 2, wherein
the superimposed image generating unit determines a type of the graphic in accordance with a type of the marker.

4. The object information acquiring apparatus according to claim 2, wherein
the superimposed image generating unit superimposes the graphic having a shape that differs from a shape of the marker onto the photoacoustic image.

5. The object information acquiring apparatus according to claim 1, wherein
an absorption coefficient of the marker at a wavelength of the light is lower than an absorption coefficient of the object at the wavelength of the light.

6. The object information acquiring apparatus according to claim 5, wherein
the photoacoustic image acquiring unit acquires the photoacoustic image derived from the photoacoustic image generated from hemoglobin in the object, and
the absorption coefficient of the marker at the wavelength of the light is at least 0.05/mm and not more than 0.1/mm.

7. The object information acquiring apparatus according to claim 1, further comprising:
an irradiating unit which irradiates the object with light from a light source;
a conversion element which receives the photoacoustic wave; and
a camera which optically captures an image of the object.

8. The object information acquiring apparatus according to claim 7, further comprising:
a measuring unit configured such that the irradiating unit, the conversion element, and the camera are arranged on a supporting member thereof; and
a scan control unit which moves the measuring unit relative to the object.

9. The object information acquiring apparatus according to claim 1, further comprising
a display unit which displays the superimposed image.

10. The object information acquiring apparatus according to claim 9, further comprising
an input unit for switching between showing and hiding the graphic in the superimposed image to be displayed on the display unit.

11. The object information acquiring apparatus according to claim 9, further comprising
an input unit for switching between types of the graphic in the superimposed image to be displayed on the display unit.

12. The object information acquiring apparatus according to claim 1, further comprising
a projector which projects the superimposed image onto the object.

13. An object information acquiring method, comprising:
acquiring an optical image obtained by optically capturing an image of an object on which a marker is arranged;
acquiring a photoacoustic image derived from a photoacoustic wave generated from the object irradiated with light; and
generating a graphic on the basis of positional information of the marker in the optical image and generating a superimposed image by superimposing the graphic onto the photoacoustic image.
